(19) United States
(12) Patent Application Publication
(10) Pub. No.: US 2016/0022171 A1
(43) Pub. Date: Jan. 28, 2016

(54) IMAGE GUIDING DEVICE FOR BRAIN SURGERY

(71) Applicant: China Medical University, Taichung (TW)

(72) Inventors: Shinn-Zong LIN, Taichung (TW); Jin-Chern CHIOU, Taichung (TW); Horng-Jyh HARN, Taichung (TW); Jeng-Ren DUANN, Taichung (TW); Yung-Jiun LIN, Taichung (TW)

(21) Appl. No.: 14/873,724

(22) Filed: Oct. 2, 2015

Related U.S. Application Data
(63) Continuation-in-part of application No. 13/615,610, filed on Sep. 14, 2012, now abandoned.

(30) Foreign Application Priority Data
Mar. 3, 2012 (TW) 101107199

(51) Int. Cl. A61B 5/06; G01R 33/28; A61B 19/00; A61B 5/00; A61B 5/055

(52) U.S. Cl. CPC A61B 5/066; A61B 5/0037; A61B 5/0042; A61B 5/055; A61B 19/203; G01R 33/285; A61B 2019/5255; A61B 2019/208

(57) ABSTRACT

An image guiding device for a brain surgery is provided. A computation processing unit receives a non-invasive basic brain image obtained prior to a surgery and plans brain surgical route data. An operating tube enters a brain according to the brain surgical route data. An image capturing module in the operating tube feeds back actual conditions of the brain in real-time. The computation processing unit performs real-time route correction to allow an operating probe to perform operations when the operating tube arrives at a target site. Thus, using the non-real-time non-invasive basic brain image collaborating with a real-time image capturing module, the computation processing unit performs route correction, thereby significantly reducing surgery risks and equipment costs.

This application is a continuation-in-part, and claims priority, from U.S. patent application Ser. No. 13/615,610 filed on Sep. 14, 2012, entitled “THE APPARATUS AND METHOD FOR USE IN SURGICAL NAVIGATION”, the entire contents of which are hereby incorporated by reference.

### FIELD OF THE INVENTION

The present invention relates to a surgical image guiding device, and particularly to an image guiding device for a brain surgery.

### BACKGROUND OF THE INVENTION

Nowadays, common human brain diseases include brain tumors, Parkinson's disease, epilepsy, etc. These diseases often easily cause a patient to involuntarily tremble, have a headache, vomit, have visual disturbance, have confusion, have body movement disability and other symptoms, thus significantly reducing the patient's life function and quality, even directly endangering the patient's life. However, when this type of patient undergoes a conservative treatment such as medication or rehabilitation, etc., and is still difficult to recover, an invasive surgery is commonly used as an ultimate treatment. A doctor has to select a very small surgical site from a patient's brain nerve, and use a surgical probe to perform a thermal ablation treatment, such as a surgical apparatus disclosed in Taiwan Patent Publication Number 200526171 entitled “APPARATUS FOR THE TREATMENT OF HOLLOW ANATOMICAL STRUCTURES”.

FIG. 1 is a schematic view showing a conventional probe surgery system. Based on the conventional invasive brain surgery, an MRI (Magnetic Resonance Imaging) image 700 is generated by nuclear magnetic resonance before surgery, and the MRI image 700 is used for establishing a virtual path plan used for positioning a positioning frame 701, and a surgical probe 702 collaborated with the positioning frame 701 is moved close to a surgical site. At this point, the surgical probe 702 returns 3D positioning signals (three-dimensional positioned location) continuously to a 3D positioning device 703, and the information of the 3D positioned location is transmitted to a master computer of the 3D positioning device 703, thus estimating the surgical site of the patient's brain and counterpointing the surgical site of the patient's brain and the CT (Computed Tomography) MRI image 700, and when the counterpointing is completed, the doctor may perform a subsequent computer-aided guided surgery. This 3D positioning may provide the doctor with the determination of the surgical site and angle, and the surgical probe 702 may be slightly adjusted in accordance with the doctor's experience.

For example, one of the conventional technology of a positioning probe includes a main body with a triangular shape and a sensor placed on each vertex of the main body, and a centre id of the main body is used for computing a three-dimensional axle center, thus generating a virtual three-dimensional space. In addition, a plurality of image positioning camera lens are installed, and a plurality of positioning measuring points are set on the probe, and thus three-dimensional data of the probe obtained from the image positioning camera lens and the virtual three-dimensional space are used to perform navigation and computation. Although the probe positioning and navigation method disclosed here may perform navigation and positioning, yet when the doctor performs an access point operation, since different people have different shapes and sizes of brains, errors are often generated due to the difference between of the virtual three-dimensional space and the surgical site. In addition, the three-dimensional positioning signal of the probe also is the possible surgical site obtained by computation.

For another example, a light ball positioning probe of branch type developed by Medtronic Company (Minnesota, USA) is to use a space area surrounded by 5 light balls to generate a virtual three-dimensional space, which also computes the three-dimensional position, and a computation error also still exists in the position confirmation operation. In addition, the light balls are the access points of passive type signals, and thus can be interfered by user differences or a sheltering effect of surrounding environment.

Taiwan Publication Number 200833293 entitled “WIRELESS POSITIONING PROBE WITH CONTINUOUS ACCESS POINT AND THE POSITIONING METHOD OF THE SAME” is presented to improve the complicated operation steps. This probe provides a probe connector with a quick release feature for fixing or releasing different types of probes, and thus the doctor may use different types of probes in accordance with different surgical needs, and also the released probes can be autoclaved for reducing the risk of infection. In addition, the probe connector can be easily dismantled and assembled without needing to use additional tools, and also can perform an angle alignment for use convenience. The positioning probe provides a functional component containing a compressed continuous access point used for selecting a continuous characteristic (this characteristic refers to the three-dimensional signals or neural interface echo) such that the operation of taking access points by using a push-button remote control can be performed without a doctor or assistant's help. Such an active sensing wireless transmitter is used for allowing the doctor to operate the probe conveniently in an operated space for transmitting the three-dimensional data.

Based on the above description, if the probe position is computed by the three-dimensional space, the surgical site is still an estimated position which is obtained indirectly, and thus the accuracy is indeed not easy to be improved. The mode of using the positioning probe to select continuous characteristic (the characteristic refers to the three-dimensional positioning or neural interface echo) is also to estimate the surgical site by computation. In addition, even if the probe can be easily dismantled and assembled without needing to use additional tools, the precise positioning is not benefited. Therefore, since the design of the conventional guided probe does not help much for performing surgery, the current surgery operated within a human body is still like operating the surgery blindly, and thus the problem that the computed three-dimensional data cannot fully meet the requirements of surgery operation still exists. Further, accumulated errors, including errors in the installation of a positioning probe, errors in operations of the positioning probe, errors generated due to a low MRI resolution, errors caused by non-real-time images, may result in a failure in arriving at the planned position.

Therefore, to solve the above issues, the U.S. Patent Publication No. 2009/0082783, “Control Unit for MRI-Guided Medical Interventional Systems”, discloses a brain surgical system based on MRI technologies. According to the above disclosure, when a patient receives the surgery, an image of the brain is updated at all times on a clinician display under a real-time MRI scanning machine. Further, with the assistance of a trajectory guide software module, surgical operations can be performed by a remote control unit. Thus, with the real-time help of the MRI system, a performer of the surgery is allowed to ensure the accuracy of the path during the surgery to arrive at the planned target site. However, such system is necessarily based on the assistance of an all-time MRI scanning system that is extremely costly. Further, not only a brain surgery frequently needs an extensive period of 10 or even 20 hours to perform, but also related surgical equipment adopting non-magnetically conductive materials that do not affect the MRI system and a special remote control unit for a doctor to operate at a remote end are required. As such, the overall system is highly complicated and costly to add up to tremendously expensive surgery costs, making such system rather unaffordable.

In a conventional common surgery, an MRI system is used for image scanning prior to the surgery to obtain the image of the brain. The surgery is planned through simulated images and a method such as the doctor's experience as previously described, and the surgery is then performed using a positioning frame and related equipment according to the plan. Take driving as an example. When driving at nighttime is expected, relying solely on a map prepared in advance for planning a driving route, and driving with only the map on hand at the time of departure without vehicle lights for observation or confirming whether road conditions ahead have changed, accidents can easily occur with even the slightest carelessness. In the technology disclosed in the abovementioned U.S. Patent Publication No. 2009/0082783, all-time MRI image scanning is applied, which is equivalent to driving with the assistance of observing a position of the vehicle and road conditions from the space according to real-time satellite images that are not only costly but inaccessible to common people. Such means is only available at an extremely high price that cannot be afforded by common people. Further, the resolution of satellite images may be inadequate for conducting driving, meaning that the driving can remain quite dangerous if vehicle lights are not turned on to observe road conditions in real-time. A target site of a brain surgery is usually several centimeters below the skull, and has a size less than 3 mm. However, the image resolution of an MRI system is only 1 mm, which obviously does not meet the patient's expectations.

### SUMMARY OF THE INVENTION

The primary object of the present invention is to lower the risk of a brain surgery under a condition of reduced costs of the brain surgery to further increase a success rate of the brain surgery.

To achieve the above object, the present invention provides an image guiding device for a brain surgery. The image guiding device for a brain surgery is applied in collaboration with a non-invasive basic brain image obtained prior to the surgery to serve as reference for pre-surgical route planning and during-surgery position guiding. The non-invasive basic brain image is obtained through scanning a head of an object by an image scanner. The head includes a skull and a brain located in the skull. The image guiding device for a brain surgery includes an computation processing unit, a positioning frame disposed at the head, an operating tube mounted and positioned on the positioning frame, an image capturing module electrically connected to the computation processing unit and disposed in the operating tube, an image display unit electrically connected to the computation processing unit, and an operating probe disposed in the operating tube.

The computation processing unit receives the non-invasive basic brain image prior to the surgery, and plans brain surgical route data from the skull to a target site of the brain. The positioning frame is positioned on the skull according to the brain surgical route data. The operating tube includes a hollow pipe, and an instrument entering end and an operating end respectively disposed at two ends of the hollow pipe. The operating end penetrates through the skull with positioning assistance of the positioning frame to enter the brain. The image capturing module includes a signal transmitting unit and a receiving unit. The signal transmitting unit transmits a probe signal at the operating end. The receiving unit receives a response signal of the brain in front of the operating end, and transmits the response signal to the computation processing unit. The computation processing unit computes the response signal to obtain a high-resolution real-time brain image having a resolution preferred than that of an MRI image. According to the real-time brain image (conditions in front of the vehicle), the MRI image (the map) and the brain surgical route plan data (the route planned in advance), the computation processing unit provides a real-time route deviation alert and a route correction suggestion at the image display unit for the user to operate the operating tube for route correction. The operating probe performs operations when the operating end of the operating tube arrives at the target site.

It is known from the above description that, the present invention provides following features.

1. Using the real-time image in front of the operating end collaborating with the non-invasive basic brain image obtained in advance as well as the brain surgical route data, a current position of the operating end can be computed by the computation processing unit during the process of the surgery, thereby providing route correction to enhance surgery precision.

2. Using the computation processing unit as well as the image capturing module, an all-time application of an MRI system can be prevented to alleviate the dependency on the MRI system, thereby reducing overall surgery costs.

### BRIEF DESCRIPTION OF THE DRAWINGS

FIG. 1 is a schematic diagram of a conventional probe system;

FIG. 2 is a block diagram of a device of the present invention;

FIG. 3A is a schematic diagram of an image capturing module according to an embodiment of the present invention;

FIG. 3B is an enlarged partial view of FIG. 3A; and

FIG. 4 is a schematic diagram of the present invention applied to a surgery.

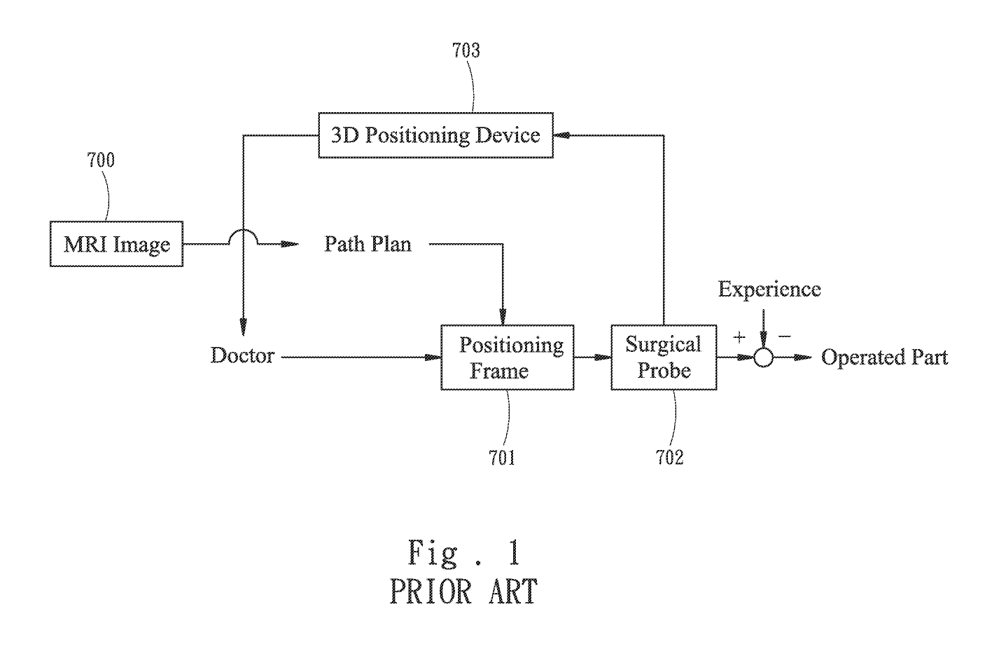

### DETAILED DESCRIPTION OF THE PREFERRED EMBODIMENTS

Details and technical contents of the present invention are given with the accompanying drawings below.

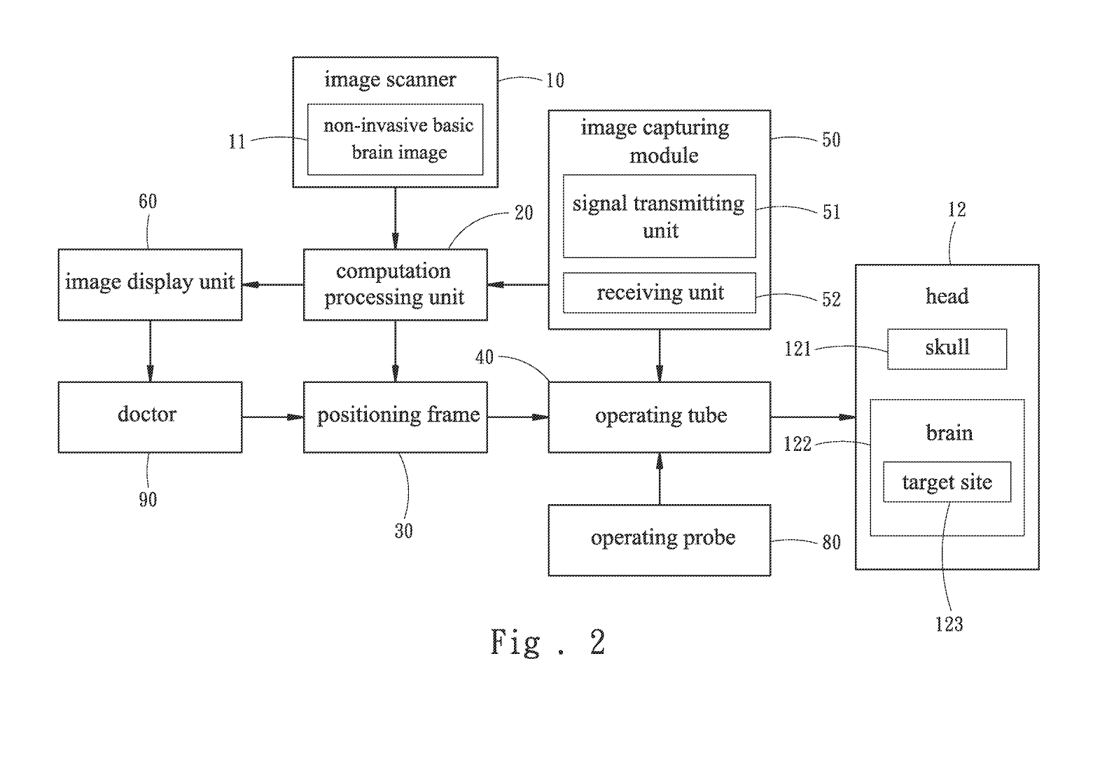

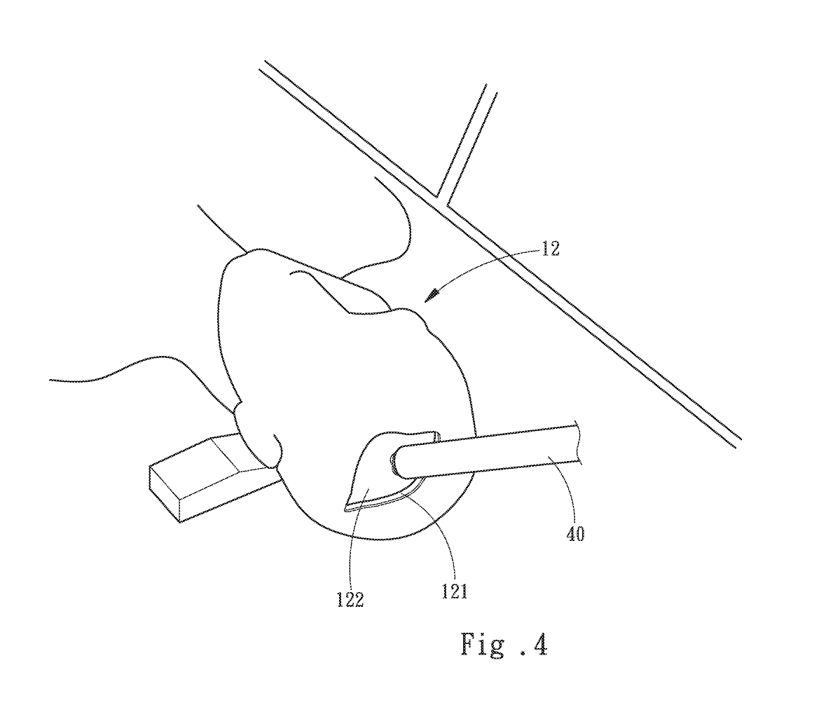

Referring to FIG. 2 and FIG. 4, the present invention provides an image guiding device for a brain surgery. The image guiding device for a brain surgery is applied in collaboration with a non-invasive basic brain image 11 obtained prior to the surgery to guide to a surgical site during the surgery. The non-invasive basic brain image 11 is obtained through scanning a head 12 of an object by an image scanner 10. More specifically, the image scanner 10 may be a magnetic resonance imaging (MRI) device that obtains the non-invasive basic brain image 11. The head 12 includes a skull 121 and a brain 122 located in the skull 121. In the present invention, an invasive surgery for the Parkinson's disease is given as an example. A doctor 90 needs to place an electrode plate(in a size of 2 mm) to one side of the hypothalamic nucleus-, and a structure deep in the brain is stimulated by electric stimulation, or heating and thermal ablation is performed on the globus pallidus or a part of the hypothalamus.

The image guiding device for a brain surgery includes a computation processing unit 20, a positioning frame 30 disposed at the head 12, an operating tube 40 positioned and operated through the positioning frame 30, an image capturing module 50 electrically connected to the computation processing unit 20 and disposed in the operating tube 40, an image display unit 60 electrically connected to the computation processing unit 20, and an operating probe 80 disposed in the operating tube 40.

The computation processing unit 20 receives the non-invasive basic brain image 11 prior to the surgery and plans brain surgery path data from the skull 121 to a target site 123 of the brain 122. More specifically, tissues of the brain 122 are formed by protein and nerve cells, which should be prevented from any kind of external invasion or damage as much as possible because brain tissues at certain positions are in control of specific body parts or logic thinking. Thus, the computation processing unit 20 may be a computer having a image software application specifically for processing brain tissues. For example, platforms such as Curve™ Image-Guided Surgery and Kick® Purely Navigation developed by BrainLab can be used to perform integration or consolidation operations on the non-invasive basic brain image 11 to identify regions to be strictly prevented from damage in the brain 122. With experiences of the doctor 90 prior to the surgery, a proceeding route from the skull 121 to the target site 123 in the brain 122 is determined and set to further plan the brain surgical route data.

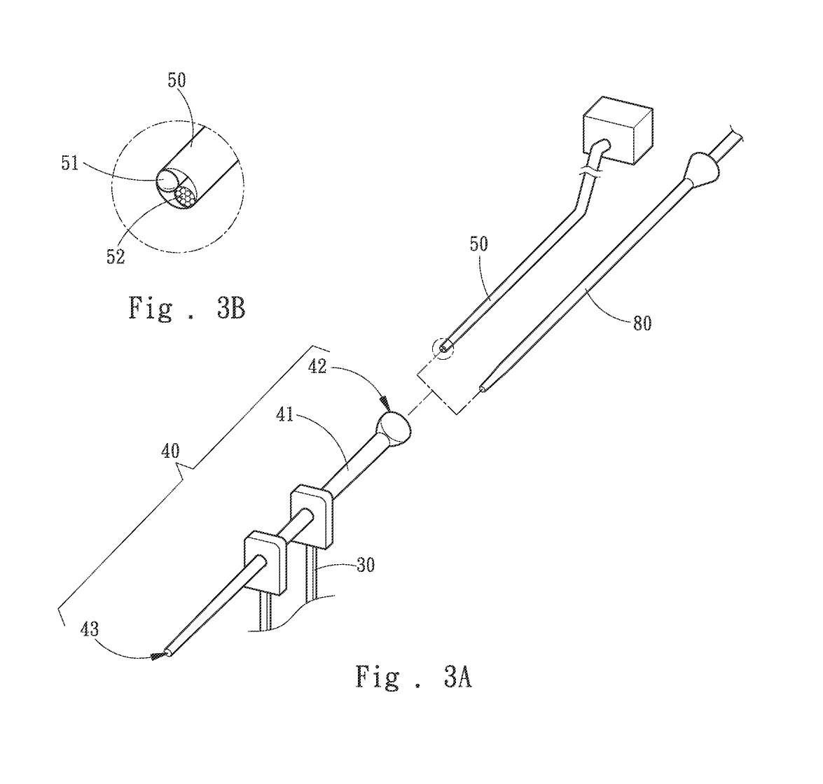

The positioning frame 30 is positioned on the skull 121 according to the non-invasive basic brain image 11 and the brain surgical route data, and a position and an angle of the operating tube 40 that enters the head 12 are positioned. The operating tube 40 includes a hollow pipe 41, and an instrument entering end 42 and an operating end 43 respectively disposed at two ends of the hollow pipe 41 (as shown in FIG. 3A and FIG. 3B). The operating end 43 penetrates through the skull 121 to enter the brain 122 with the positioning assistance of the positioning frame 30. It should be noted that, the hollow pipe 41 may be a straight pipe having flexibility, and is capable of bypassing specific areas of the brain 122 in a non-linear manner.

Referring to FIG. 3A and FIG. 3B, the image capturing module 50 includes a signal transmitting unit 51 and a receiving unit 52. The signal transmitting unit 51 sends a probe signal at the operating end 43, and the receiving unit 52 receives a response signal of the brain 122 and transmits the response signal back to the computation processing unit 20. The signal transmitting unit 51 is an optical transmitter. The probe signal is an optical signal and is received by the receiving unit 52 through reflection/diffraction effects of illumination of light. In the present invention, such signal is referred to as the response signal. By processing the response signal with the computation processing unit 20, a tissue surface image, a three-dimensional structure image, or a four-dimensional dynamic structural image is presented. The method of transmitting the above signals may be performed by optical fibers. Alternatively, the response signal may be obtained through other types of electromagnetic wave reflection/diffraction methods. For example, the image capturing module 50 may capture an image using optical coherence tomography (OCT), which provides a resolution in a micrometer scale, i.e., the 100 to 1000 times of the MRI resolution.

More specifically, the image capturing module 50 may enter the brain 122 along with the operating tube 40, and the operating tube 40 may halt at a plurality of checkpoints (not shown) planned in the brain surgical route data to obtain the response signals of these checkpoints through the image capturing module 50.

The computation processing unit 20 obtains a real-time image in front of the operating end 43 by computing the real-time response signal of the brain 122, and corrects surgical route data in collaboration with the non-invasive basic brain image 11 and the brain surgical route data planned prior to the surgery. That is, a real-time route deviation alert and a path correction suggestion may be provided to the image display unit 60 for a user (or the doctor 90) to operate the operating tube 40 for route correction. It should be noted that, as a result of factors of the skull 121 being opened, loss of blood and anesthesia during the surgery, the pressure of the brain 122 can be changed, hence leading to issues of brain deformation, drifts of the plurality of checkpoints, and a drift in the position of the target site. As the non-invasive basic brain image 11 is an image of the brain 122 obtained prior to the surgery, due to the above reasons, the computation processing unit 20 may compare the real-time image obtained in front of the operating end 43 with the non-invasive basic brain image 11 to determine the current relative position of the operating end 43 of the operating tube 40 in the brain 122, thereby correcting the estimated position according to the actual position. Because the surgery on the brain 122 requires an extremely high precision, the error between the actual route and the simulated route may need to be smaller than 1 mm. Thus, correction on the route of the image capturing module 50 needs to be performed in order to ensure the accuracy and precision of the route.

The operating probe 80 performs operations when the operating end 43 of the operating tube 40 arrives at the target site 123, with details described below. When the operating probe 80 enters the brain 122 along with the operating tube 40, the operating probe 80 also stays on-call in the hollow pipe 41, and performs operations when arriving at the target site 123. In the example of treating the Parkinson's disease in the embodiment, when the target site 123 is reached, a stimulation electrode is implanted or heating and thermal ablation is performed. Further, functional operations include measurement, stimulation, release or clamping may also be performed.

In conclusion, the present invention provides following features.

1. Using the real-time image in front of the operating end collaborating with the non-invasive basic brain image obtained in advance as well as the brain surgical route data, a current position of the operating end can be computed by the computation processing unit during the process of the surgery, thereby providing route correction to enhance surgery precision.

2. Using the computation processing unit as well as the image capturing module, an all-time application of an MRI system can be prevented to alleviate the dependency on the MRI system, thereby reducing overall surgery costs.

3. Taking an example of driving, the present invention performs route planning (the brain surgical route data) of navigation software in the dark, and stays aware of road conditions ahead at all times with lighting equipment (the image capturing module) of the vehicle. Thus, not only the issue of being unaware of actual road conditions and solely depending on a navigation system when driving in the dark can be prevented, but the issue of having to learn actual road conditions through information such as expensive satellite information having an inadequate resolution can be eliminated.

What is claimed is:

1. An image guiding device for a brain surgery, applied in collaboration with a non-invasive image brain image obtained prior to a surgery to guide a surgical position during the surgery, the non-invasive image brain image obtained through scanning a head of an object by an image scanner, the head comprising a skull and a brain located in the head, the image guiding device for a brain surgery comprising:
a computation processing unit, receiving the non-invasive image brain image, and planning a brain surgical route datum from the skull to a target site of the brain prior to the surgery;
a positioning frame, positioned on the skull according to the non-invasive image brain image and the brain surgical route data;
an operating tube, mounted and positioned on the positioning frame, comprising a hollow pipe, and an instrument entering end and an operating end respectively disposed at two ends of the hollow pipe, the operating end penetrating through the skull to enter the brain with positioning assistance of the positioning frame;
an image capturing module, electrically connected to the computation processing unit and disposed in the operating tube, comprising a signal transmitting unit and a receiving unit, the signal transmitting unit sending a probe signal at the operating end, the receiving unit receiving a response signal of the brain and transmitting the response signal to the computation processing unit for computation to obtain a real-time image;
an image display unit, electrically connected to the computation processing unit, the computation processing unit computes the real-time response signal of the brain to obtain the real-time image in front of the operating end, collaborating with the non-invasive basic brain image and the brain surgical route datum to correct surgical route data, and displaying the real-time image at the image display unit for a user to operate the operating tube to perform route correction; and
an operating probe, disposed in the operating tube, performing operations when the operating end of the operating tube arrives at the target site.

2. The image guiding device for a brain surgery of claim 1, wherein the image scanner is a magnetic resonance imaging (MRI) device that obtains the non-invasive basic brain image.

3. The image guiding device for a brain surgery of claim 1, wherein the signal transmitting unit is an optical transmitter, and the probe signal is an optical signal.
